(19) United States
(12) Patent Application Publication
(10) Pub. No.: US 2010/0022846 A1
(43) Pub. Date: Jan. 28, 2010

(54) Method for Health Monitoring

(76) Inventors: Philippe Pouletty, Paris (FR); Daniel Cohen, Le Vesinet (FR)

Correspondence Address: NIXON & VANDERHYE, PC, 901 NORTH GLEBE ROAD, 11TH FLOOR, ARLINGTON, VA, 22203, US

(21) Appl. No.: 12/085,598

(22) PCT Filed: Nov. 28, 2006
(86) PCT No.: PCT/EP2006/068978

§ 371 Date: Sep. 22, 2009

Related U.S. Application Data
(60) Provisional application No. 60/739,924, filed on Nov. 28, 2005.

(51) Int. Cl. A61B 5/00; G06F 17/30

(52) U.S. Cl. 600/300; 707/102; 707/E17.044; 707/104.1; 707/E17.019

(57) ABSTRACT

This invention relates to methods for monitoring the anatomy of an individual, which methods comprise the steps of: (i) subjecting a healthy or asymptomatic individual to non-invasive analysis techniques, such as imaging or acoustic techniques, to obtain images or data on determined cells, tissue(s) or organs, at different time points over a period of more than one month, preferably over years; (ii) collecting the images or data in a database; (iii) comparing the images or data to the images or data previously acquired for the same individual, in essentially the same conditions; wherein said comparison of images or data obtained at different time points allows definition of non-pathological baseline anatomy for this individual.

The present invention relates to databases of imaging or sonic or ultrasonic data for a single subject over months or years to detect tissue or organ pathological variations within a subject with greater accuracy than by using population-based comparisons.

Currently, diagnosis of pathological conditions using imaging techniques or acoustic techniques relies on non-frequent testing of a given individual, mostly at times of symptoms or for aging individuals. Data are then analysed per “normal” standards based on images collected from hundreds or thousands of other, unrelated, individuals.

However, because of large subject-to-subject variability of tissues and organs in the general healthy and diseased populations, definition of “normality” comprises major differences of macroscopic and microscopic anatomy, and identification of abnormality for a given individual requires either very large data bases of control subjects and very complex algorithms, or advanced stage of disease to distinguish abnormal from normal or subjective interpretation by a trained health professional.

The inventors herein describe a new preventive health technique based on repeat imaging of a single individual healthy patient over months or years to provide intra-individual baseline data. The inventors propose to name this system: The Health Arch Systems.

A method for monitoring the anatomy of an individual, which method comprises the steps of
(i) repeatedly subjecting a healthy or asymptomatic individual to non-invasive analysis techniques, such as imaging or acoustic techniques, to obtain images or data on determined cells, tissue(s) or organs, at different time points over a period of more than one month, preferably over years;
(ii) collecting the images or data in a database;
(iii) comparing the images or data to the images or data previously acquired for the same individual, in essentially the same conditions;
   wherein said comparison of images or data obtained at different time points allows definition of non-pathological baseline anatomy for this individual. Such method allows the monitoring of the health of an individual, insofar as a difference between the newly acquired images or data as compared to baseline images or data is potentially indicative of an abnormality or disease that has developed.

The non-invasive analysis techniques include X-ray, Positron Tomography, Single Photon Emission Computed Tomography, X-ray Computerized Tomography, Magnetic Resonance Imaging, ultrasonography, Doppler techniques, high frequency ultrasounds, diffusion tensor imaging or any technique providing a difference of image, contrast, density or sound, between different cells, tissues or organs, whether 2 Dimensional or 3 Dimensional.

The images or data may be collected under various physiological conditions, such as different body positions, different body conditions (e.g. full or partially full or empty bladder, stomach, lungs, menstrual cycle, rest or stress).

Furthermore, these individual databases can be refined if taking into account other type of data, such as family data and/or using family members to further analyse individual physiological variability for a given subject. The medical history and laboratory data can be taken into account too, as well as the environmental factors, or the presence of phenotypic or genotypic markers of a disease. A molecular profiling (by analysis of DNA, RNA, or proteins) of the individual may indeed be useful to correlate with the variation of images or data recorded.

The method may be easy to implement, by using an instrument-based technique to monitor human microscopic or macroscopic anatomy or physiology, that may be installed on a long term basis for acquisition of data in the personal house or workplace or school of a given individual in order to generate a database of individual anatomical or physiological data over time.

The invention further provides a computer-readable medium having stored thereon instructions for a computer to access the medium comprising:
instructions to access data on the medium;
a main database on the medium related to images or data obtained by non-invasive analysis techniques, such as imaging or acoustic techniques, on determined tissues of an individual, at a given time and under given conditions;
the computer-readable medium including instructions to compare the updated images or data of the main database to the images and data previously stored; wherein said comparison of images or data obtained at different time points allows definition of non-pathological baseline anatomy for this individual. The comparison allows to highlight a difference in the images or the data that is indicative of a potential abnormality or disease that has developed or is developing.

This computer-readable medium may further comprise:
additional database(s) on the medium respectively related to (a) other phenotypic data of the individual; (b) genotypic data of the individual; (c) health history and condition of family members; and/or (d) environmental factors;
and instructions to analyse the main database in correlation with the additional database(s).

Algorithms and computer-based comparison of data can be developed to provide for low-cost automated detection of potentially pathological conditions. The data or images can be compressed or processed, for example using wavelets techniques or other signal processing methods. The identification of individuals and security of data can be ensured by any state of the art process.

The ability to monitor a same subject for months or years prior to development of a pathological condition, and monitoring the subject tissues and organs under multiple physiological conditions (body position, resting or stress condition, monthly cycle, youth growth . . . ) shall ensure a more accurate and/or earlier and/or lower cost diagnosis using imaging or sound techniques (or other non-invasive technique for analysing anatomy), by defining “normality” using the subject as his own control. Using this approach, normality falls in a much narrower range of anatomical and test data, and a small variation in the future may be identifiable earlier or more accurately than by using population-based standards.

For example, blood vessels can significantly vary in size, length, anatomy within a population, making identification of an early stage aneurysm or stenosis difficult when relying on population-based standards. For example, lung, liver, kidney, heart, brain, breast, colon, pancreas, thyroid, stomach, bladder, prostate, testis, uterus, tooth anatomy as well as other tissue and organ anatomy varies significantly from normal subject to normal subject, making a single diseased patient at an early stage likely to fall within normal standards according to population based-analysis. In contrast, this anatomy is less variable (subject to taking into account normal organ growth and physiological variations) within a same individual. Therefore, building individual data bases over at least several months or years with at least 3 (over at least 6 months) or better 4-10 data points over several years for a given individual provides accurate historical controls to identify a sudden or small anatomical or pathological change for this individual.

1.-17. (canceled)

18. A method for monitoring the health of an individual, which method comprises the steps of:
(i) repeatedly subjecting a healthy or asymptomatic individual to non-invasive analysis techniques to obtain images or data of several distinct types of determined cells, tissue(s) or organs, at different time points over a period of more than one month;
(ii) collecting the images or data in a database;
(iii) comparing the images or data to images or data previously acquired for the same individual, in essentially the same conditions;
wherein said comparison of images or data obtained at different time points allows definition of non-pathological baseline images or data for this individual.

19. The method of claim 18, wherein a difference between newly acquired images or data as compared to baseline images or data is potentially indicative of an abnormality or disease that has developed.

20. The method of claim 18, wherein the images or data are collected under various physiological conditions.

21. The method of claim 18, wherein the images or data are collected using imaging or acoustic techniques.

22. The method of claim 18, wherein the images or data are collected at different time points over years.

23. The method of claim 18, wherein the images or data undergo processing techniques to facilitate storage, improve quality or analysis.

24. The method of claim 18, wherein wavelet signal processing techniques are applied to data.

25. The method of claim 18, wherein 3 Dimensional images are analysed.

26. The method of claim 18, wherein data obtained under different body positions are compared and define boundaries of non pathological variability for a single individual.

27. The method of claim 18, wherein data obtained under different physiological conditions are compared and define boundaries of non pathological variability for a single individual.

28. The method of claim 18, wherein data obtained at time intervals during growth of a child are used to predict normal anatomy upon adulthood.

29. The method of claim 18, wherein the images or data from at least two different imaging techniques are combined or compared to improve accuracy.

30. The method of claim 18, wherein the analysis of images and data further takes into account the health history and condition of family members.

31. The method of claim 18, wherein the collected images or data are further correlated to genotypic or phenotypic features of the individual.

32. The method of claim 18, wherein the collected images or data are further correlated to environmental factors.

33. The method of claim 18, wherein step (i) is performed using an instrument-based technique to monitor human microscopic or macroscopic anatomy or physiology, that is installed over several months or years for acquisition of data in the personal house or workplace or school of a given individual in order to generate a database of individual anatomical or physiological data over time.

34. A computer-readable medium having stored thereon instructions for a computer to access the medium comprising:
instructions to access data on the medium;
a main database on the medium related to images or data obtained by non-invasive analysis techniques, such as imaging or acoustic techniques, of several distinct types of determined tissues of an individual, at a given time point and under given conditions;
the computer-readable medium including instructions to compare the updated images or data of the main database to the images and data previously stored; wherein said comparison of images or data obtained at different time points allows definition of non-pathological baseline anatomy for this individual.

35. The computer-readable medium of claim 34, further comprising
additional database(s) on the medium respectively related to (a) other phenotypic data of the individual; (b) genotypic data of the individual; (c) health history and condition of family members; and/or (d) environmental factors;
and instructions to analyse the main database in correlation with the additional database(s).

36. A computer-readable medium having stored thereon instructions for a computer to access the medium comprising:
instructions to access data on the medium;
a main database on the medium related to images or data obtained by non-invasive analysis techniques, such as imaging or acoustic techniques, on determined tissues of an individual, at a given time point and under given conditions;
the computer-readable medium including instructions to compare the updated images or data of the main database to the images and data previously stored; wherein a difference between the newly acquired images or data as compared to baseline images or data is potentially indicative of an abnormality or disease that has developed.

37. A method of producing a subject database, the method comprising the steps of:
(i) repeatedly subjecting a healthy or asymptomatic individual to non-invasive analysis techniques to obtain images or data of several distinct types of determined cells, tissue(s) or organs, at different time points over years; and collecting the images or data in a database.
